SEQUENCE LISTING (18 sequences)

SEQ ID NO: 1 - DNA, 24 bp, Artificial Sequence
  chemically synthesized
aaagaaagctcatggcccaaccac

SEQ ID NO: 2 - DNA, 25 bp, Artificial Sequence
  chemically synthesized
gttgttccttactgttagacgggtg

SEQ ID NO: 3 - DNA, 27 bp, Artificial Sequence
  chemically synthesized
aataaccattggatcaatctgtctggt

SEQ ID NO: 4 - DNA, 29 bp, Artificial Sequence
  chemically synthesized
aattgccggttaatatcactgaagttgtg

SEQ ID NO: 5 - DNA, 24 bp, Artificial Sequence
  chemically synthesized
acttcccggaaatgacaacagcac

SEQ ID NO: 6 - DNA, 24 bp, Artificial Sequence
  chemically synthesized
actgagggtctcccaatagagcat

SEQ ID NO: 7 - DNA, 24 bp, Artificial Sequence
  chemically synthesized
acgattggctctgtttctctcacc

SEQ ID NO: 8 - DNA, 24 bp, Artificial Sequence
  chemically synthesized
ccttctctatggtggtgttggtca

SEQ ID NO: 9 - DNA, 24 bp, Artificial Sequence
  chemically synthesized
acaaaggtgtaacggcagcatgtc

SEQ ID NO: 10 - DNA, 30 bp, Artificial Sequence
  chemically synthesized
tgcattctgatagagactttgttggtcagc

SEQ ID NO: 11 - DNA, 23 bp, Artificial Sequence
  chemically synthesized
accattggttcggtctgtatgac

SEQ ID NO: 12 - DNA, 24 bp, Artificial Sequence
  chemically synthesized
cagcagcaaagttggtgttgctga

SEQ ID NO: 13 - DNA, 208 bp, Influenza A virus
aaagaaagctcatggcccaaccacaacacaaacggagtaacggcagcatgctcccatgaggggaaaagcagtttttacagaaatttgctatggctgacggagaaggagggctcatacccaaatctgaaaaattcttatgtgaacaaaaaagggaaagaagtccttgtactgtggggtattcatcacccgtctaacagtaaggaacaac

SEQ ID NO: 14 - DNA, 221 bp, Influenza A virus
acttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtggaatatgcgacagtcctcatcagatccttgatggagaaaactgcacactaatagatgctctattgggagaccctcagt

SEQ ID NO: 15 - DNA, 200 bp, Influenza A virus
acaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgca

SEQ ID NO: 16 - DNA, 200 bp, Influenza A virus
aataaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaatt

SEQ ID NO: 17 - DNA, 202 bp, Influenza A virus
acgattggctctgtttctctcaccatttccacaatatgcttcttcatgcaaattgccatcttgataactactgtaacattgcatttcaagcaatatgaattcaactcccccccaaacaaccaagtgatgctgtgtgaaccaacaataatagaaagaaacataacagagatagtgtatctgaccaacaccaccatagagaagg

SEQ ID NO: 18 - DNA, 205 bp, Influenza A virus
accattggttcggtctgtatgacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctg

(19) United States
(12) Patent Application Publication
(10) Pub. No.: US 2012/0015346 A1
(43) Pub. Date: Jan. 19, 2012

(54) INFLUENZA VIRUS DETECTION AND DIAGNOSIS

(75) Inventors: Manojkumar Ramanunninair, Ossining, NY (US); Doris J. Bucher, New York, NY (US)

(73) Assignee: New York Medical College, Valhalla, NY (US)

(21) Appl. No.: 13/179,210

(22) Filed: Jul. 8, 2011

Related U.S. Application Data
(60) Provisional application No. 61/362,412, filed on Jul. 8, 2010.

(51) Int. Cl. C12Q 1/70; B65D 85/00; C07H 21/04

(52) U.S. Cl. 435/5; 536/24.33; 206/525

(57) ABSTRACT

The invention discloses compositions comprising nucleic acid(s) for rapid detection, identification, differentiation, and/or diagnosis of certain Influenza virus A subtypes, e.g., H1N1, H3N2 and A(2009 H1N1)pdm, and methods of use thereof, e.g., Short-run RT-PCR, including an RT-PCR assay kit, comprising the disclosed composition(s).

This application claims the benefit, under 35 USC Section 119, of U.S. Provisional Appl. No. 61/362,412 filed Jul. 8, 2010, the disclosure of which is hereby incorporated herein by reference.

### FIELD OF THE INVENTION

The invention relates to compositions comprising nucleic acid(s), e.g., oligonucleotide primer(s), probe(s), for rapid detection, identification, differentiation, and diagnosis of certain Influenza virus A subtypes, e.g., H1N1, H3N2 and A(2009 H1N1)pdm, and methods of use thereof, e.g., nucleic-acid based method(s). The invention further relates to assays and test, and an article of manufacture, e.g., assay, test and/or diagnostic kit, comprising the compositions described herein, and a method of manufacturing and using said article.

### BACKGROUND OF THE INVENTION

Influenza virus is a single stranded, negative sense RNA virus, which belongs to the family Orthomyxoviridae. There are three known types of influenza viruses—A, B and C. Type A influenza viruses are further classified into subtypes based on antibody responses to virus surface glycoproteins, Hemagglutinin (HA) and Neuraminidase (NA). These different types of HA (16 known) (Fouchier et al., 2005. Characterization of a novel influenza A virus hemagglutinin subtype (H16) obtained from black-headed gulls. J. Virol., 79(5), 2814-2822) and NA (9 known) form the basis of the H and N distinctions, for example H1N1.

Among several known influenza A virus subtypes, influenza H1N1 and H3N2 subtypes have been prevalent in the human population, and are most commonly associated with “seasonal” flu. Influenza viruses have been observed to circulate in nearly every part of the world. Influenza A virus causes yearly epidemics in certain populations, e.g., those having partial immunity to the virus. Illness (i.e., “flu” and its associated symptoms) result in hospitalizations and deaths, particularly among high-risk groups, e.g., the very young, elderly (e.g., age 65 or older), immunocompromised, or chronically ill individuals. Worldwide, these annual epidemics result in about 3 to 5 million cases of severe illness, and about 250,000 to 500,000 deaths. They also have the potential to cause pandemics (pdm) as occurred in 1918, 1957, 1968 and 2009.

In addition to humans, Influenza A viruses are also found in many different animals, including ducks, chickens, pigs, horses, dogs, whales, and seals. In some instances, genes from Influenza viruses from different species (e.g., duck and humans) can mix to create a new virus (i.e., “antigenic shift”). Antigenic shift results when a new influenza A subtype, to which most people have little or no immunity, infects humans. One such example is the 2009 novel swine-origin influenza A virus [A(2009 H1N1)pdm] subtype, commonly referred to as “swine flu,” which caused what was declared by WHO as the first influenza pandemic of the 21^{st} century. The virus appeared to be a new strain of H1N1 which resulted when a previous triple reassortment of bird, pig, and human flu viruses further combined with a Eurasian pig flu virus (Trifonov et al., 2009. Geographic dependence, surveillance, and origins of the 2009 influenza A (H1N1) virus. New Eng. J. Med. 61, 115-119).

Conventional methods for laboratory identification of human influenza virus infections are commonly performed using immunoassays (e.g., to detect viral antigen); viral neuraminidase activity; virus isolation in cell culture; or detection of influenza-specific RNA, e.g., by reverse transcriptase polymerase chain reaction (RT-PCR). These tests differ in their sensitivity and specificity in detecting influenza viruses as well as in their commercial availability, the amount of time needed from specimen collection until results are available, and the tests' ability to distinguish between different influenza virus types (A versus B) and influenza A subtypes (e.g., novel H1N1 (e.g., A(2009 H1N1)pdm) versus seasonal H1N1 versus seasonal H3N2).

In recent years commercial rapid influenza diagnostic tests (RIDTs) have become available that can provide results within 30 minutes or less, however these are predominantly antigen detection based methods. The advantage of RIDTs is that results become available in a clinically relevant time period to inform clinical decisions. Further, some RIDTS have been applied as “point-of-care” tests, i.e., they can be used in locations outside of a central laboratory e.g., at the patient bedside, in a doctor's office, or in the field.

However, there are some shortcomings associated with use of presently-available commercial RIDTs. For example, their wide availability has resulted in their increasing application to clinical situations which may be inappropriate or where scientific data are lacking Additionally, while their specificity is high, median 90-95% (http://www.who.int/csr/disease/avian_influenza/guidelines/rapid_testing/en/index.html) their sensitivity is variable, with a median 70-75%, which is lower than that of cell culture. Further, while some commercial RIDTs can detect and distinguish between influenza A and B viruses, none of the currently FDA approved RIDTs can distinguish between influenza A virus subtypes (e.g., seasonal influenza A (H1N1) versus seasonal influenza A (H3N2) viruses).

For example, Hurt et al., 2009 studied the performance of influenza rapid “point-of-care” antigen tests for A(H1N1)pdm influenza viruses and reported that the tests are significantly less sensitive than conventional PCR assays and recommended that negative results should be verified by PCR test (Hurt et al., 2009. Performance of influenza rapid point-of-care tests in the detection of swine lineage A(2009 H1N1) influenza viruses. Influenza Other Respi. Viruses, 3(4), 171-176).

RT-PCR assays detect influenza virus RNA extracted from viable and non-viable virus or freshly extracted or stored RNA and are more sensitive than cell culture with improved detection rates over cell culture between 2-13% (http://www.who.int/csr/disease/avian_influenza/guidelines/rapid_testing/en/index.html). However, a disadvantage of currently published conventional RT-PCR tests, for influenza virus detection, is that they require a total of 3.5 to 5 hours to obtain final confirmatory results (e.g., >2.5 hours reaction time with an additional 30 minutes to 1 hour for detecting/analyzing the reaction product). For example, Phipps et al. (2004) reported an RT-PCR assay which requires a single set of primers, based on conserved HA coding sequences for genetic identification of influenza A viruses which requires 2 hours and 36 minutes reaction time (Phipps. et al., 2004. Genetic subtyping of influenza A viruses using RT-PCR with a single set of primers based on conserved sequences within the HA2 coding region. J. Virol. Methods, 122, 119-122); Chan et al., 2006, developed an RT-PCR assay for the identification and differentiation of seasonal H1N1 and H3N2 viruses, which required a reaction time of 5 hours (Chan et al. 2006. Amplification of the entire genome of influenza A virus H1N1 and H3N2 subtypes by reverse-transcription polymerase chain reaction. J. Virol. Methods, 136, 38-43).

The currently recommended methods for confirming A(2009 H1N1)pdm are nucleic acid testing using real-time reverse transcriptase polymerase chain reaction (rRT-PCR) and viral culture. While real time RT-PCR tests have gained Emergency Use Authorization (EUA) from the U.S. Food and Drug Administration (FDA), the disadvantage is that they require expensive laboratory equipment and highly trained personnel. Further, while rapid identification can be performed using real time RT-PCR assays, because of the above challenges vis-à-vis cost and skilled personnel, they do not permit application to point-of-care tests, e.g., at the patient bedside or in a doctor's office.

For example, Poon et al. (2009) developed a one-step real time RT-PCR based on HA and M genes which requires a reaction time of 2 hours and 30 minutes for detection of A(2009 H1N1)pdm but is not capable of detecting seasonal H1N1 or H3N2 viruses. (Poon et al., 2009. Molecular detection of a novel human influenza virus (H1N1) of pandemic potential by conventional and real-time quantitative RT-PCR assays. Clin. Chem., 55, 1555-1558). Yang et al. (2009) reported rapid SYBR green real time RT-PCR assays to identify influenza seasonal H1N1 and 2009 H1N1 pandemic strains, but not H3N2 viruses, requiring a reaction time of 80 minutes. (Yang, et al., 2009. Rapid SYBR Green I and modified probe real-time RT-PCR assays identify influenza H1N1 viruses and distinguish between pandemic and seasonal strains. J. Clin. Microbiol., DOI:10.1128/JCM.01646-09.) The United States Centers for Disease Control (CDC) developed a real time RT-PCR for the identification of A(2009 H1N1)pdm viruses based on HA amplification in 66 minutes (http://www.who.int/cseresources/publications/swineflu/realtimeptper/en/index.html). Whiley et al., 2009, reported a real time RT-PCR assay for the identification of A(2009 H1N1)pdm viruses based on HA and NA genes requiring a reaction time of 91 minutes, however, this assay did not permit differentiation of A(2009 H1N1)pdm from seasonal H1N1 and H3N2 viruses. (Whiley et al., 2009. Detection of novel influenza A(H1N1) virus by real-time RT-PCR. J. Clin. Virol. 45, 203-204).

Another approach in identifying influenza virus requires prior amplification of the virus in cell culture, an additional 48 hours before analysis, along with the use of sophisticated chemistry. For example, Daum et al. (2002) optimized RT and multiplex PCR in a single step with a reaction time of 90 minutes for simultaneously typing, and sub-typing of influenza viruses in cell culture. (Daum et al., 2002. A rapid single-step multiplex reverse transcription-PCR assay for the detection of human H1N1, H3N2, and B influenza viruses. J. Clin. Virol. 25, 345-350).

Thus, existing methodologies, including currently known RT-PCR-based methods, have short comings vis-à-vis sensitivity, specificity (e.g., distinguishing between different influenza virus types and subtypes), length of time needed from clinical specimen collection until final confirmatory results can be obtained (e.g., 3-5 hours), and require expensive laboratory equipment as well as highly trained personnel, and therefore do not readily permit application to point-of-care tests, e.g., at the patient bedside or in a doctor's office. A summary of currently available laboratory influenza diagnostic tests can be found at the website maintained by the United States Centers for Disease Control and Prevention (www.cdc.gov).

Therefore, there is a pressing need in the art for approaches which are efficacious with respect to specificity and sensitivity as well as in terms of cost, skill, labor, and time to detect, identify, diagnose, and distinguish influenza virus A subtypes. Early and efficient diagnosis can avert potentially devastating (including fatal) outcomes of the disease, e.g., by aiding in making informed treatment decisions, thus underscoring the need for rapid, point-of-care tests which satisfy the above requirements, while overcoming the challenges of existing methods.

The above stated needs are met by the present invention which, in one aspect, relates to a Short-run RT-PCR assay that can be used to rapidly detect, identify as well as effectively distinguish Influenza A virus subtypes seasonal H1N1, H3N2 and A(2009 H1N1)pdm. The advantages of the present method(s) are that it is cost-effective, does not require expensive equipment, can be performed in laboratories or clinics having basic facilities to perform PCR tests, and requires a short reaction time (i.e., Short-run), e.g., 45 minutes, 60 minutes, 75 minutes, and up to 90 minutes to obtain final confirmatory results. Further, in view of the aforementioned advantages, the method disclosed herein has potential for application to point-of-care diagnostic tests. Additionally, the methods are useful in epidemiological (e.g., global surveillance of epidemic, pandemic, and identification of hitherto unknown re-assorted viruses) of influenza A virus subtypes discussed herein, including novel combinations thereof, as well as in research studies directed toward development of prophylactic and therapeutic approaches.

### SUMMARY OF THE INVENTION

In one aspect, the invention relates to isolated and purified nucleic acids, e.g., polynucleotides (e.g., oligonucleotide primers and/or probes) consisting of sequences, including mutation(s) thereof, and complementary sequence(s) thereof corresponding to certain conserved genomic regions (e.g., from HA and NA genes) of certain, influenza A virus subtypes, for example, H1N1, H3N2 and A(2009 H1N1)pdm.

In another aspect, the invention relates to composition(s) comprising the above nucleic acid(s), including mixtures thereof, accessory reagents (e.g., enzymes, nucleotides) for detecting, and/or identifying, and/or diagnosing specific influenza virus A subtypes, e.g., seasonal H1N1, seasonal H3N2 and A(2009 H1N1)pdm viruses, in a sample, e.g., biological sample.

In another aspect, the invention relates to method(s) comprising the above nucleic acid(s) and/or composition(s) for detecting, and/or identifying, and/or diagnosing specific influenza virus A subtypes, e.g., seasonal H1N1, seasonal H3N2 and A(2009 H1N1)pdm viruses, in a sample, e.g., biological sample.

In another aspect, the invention relates to preparing a biological sample, e.g., a laboratory specimen and/or obtaining a suitable clinical specimen, from a subject in need thereof, and/or isolating and/or purifying biological material from a biological sample, and testing the sample according to the methods described herein, for detecting, and/or identifying, and/or diagnosing specific influenza virus A subtypes, e.g., seasonal H1N1, seasonal H3N2 and A(2009 H1N1)pdm viruses, in said sample.

In another aspect, the invention relates to an RT-PCR based method, comprising the above composition(s) of the invention, for detecting, and/or identifying, and/or diagnosing specific influenza virus A subtypes, e.g., seasonal H1N1, seasonal H3N2 and A(2009 H1N1)pdm viruses, in a sample, e.g., biological sample.

In another aspect, the invention relates to a Short-run RT-PCR assay comprising the above composition(s) of the invention, for detecting, and/or identifying, and/or diagnosing specific influenza virus A subtypes, e.g., seasonal H1N1, seasonal H3N2 and A(2009 H1N1)pdm viruses, in a sample, e.g., biological sample, wherein the assay can be performed in a short period of time e.g., 45-90 minutes in the laboratory, doctor's office or in the field.

In another aspect the invention relates to use of said Short-run RT-PCR assay in identifying the origin of parental genes in an Influenza A virus vaccine candidate(s), e.g., in a high-yield reassortant vaccine preparation.

In another aspect, the invention relates to a rRT-PCR assay comprising the above composition(s) of the invention, for detecting, and/or identifying, and/or diagnosing specific influenza virus A subtypes, e.g., seasonal H1N1, seasonal H3N2 and A(2009 H1N1)pdm viruses, in a sample, e.g., biological sample.

In another aspect, the invention relates to an RT-PCR based method comprising the above composition(s) of the invention, for detecting, and/or identifying, and/or diagnosing specific influenza virus A subtypes, e.g., seasonal H1N1, seasonal H3N2 and A(2009 H1N1)pdm viruses, in a sample, e.g., biological sample, in a single reaction mix (e.g., multiplex PCR).

In yet another aspect, the invention relates to a packaged article, e.g., an article of manufacture, such as an assay and/or diagnostic kit, comprising the composition(s) of the invention, optionally with a label(s) and/or with instructions for use. Such label(s) include(s) ingredients, amounts or dosages, and/or indications. Such instructions include directing or promoting, including advertising, use of said article of manufacture.

In a further aspect, the invention relates to a method of manufacturing an article of manufacture comprising any of the compositions of the invention described herein, packaging the composition to obtain an article of manufacture and instructing, directing or promoting the use of the article of manufacture for any of the uses described herein. Such instructing, directing or promoting includes advertising.

### BRIEF DESCRIPTION OF THE DRAWINGS

FIG. 1. Detection of Influenza A viruses HA by Short-run RT-PCR amplification at 550 C and 610 C (annealing temperatures). M-100 bp Molecular weight marker; Lanes 1 & 4-seasonal H1N1 virus at 55° C. and 61° C., respectively; Lanes 2 & 5-H3N2 virus at 55° C. and 61° C., respectively; Lanes 3 & 6 A(2009 H1N1)pdm at 55° C. and 61° C., respectively.

FIG. 2. Detection and differentiation of HA and NA genes of influenza A positive control viruses by subtype specific primers in Short run RT-PCR assay. Lane L-100 bp Molecular weight marker; Lane 1-H1N1 virus with H1N1 specific HA primer; Lane 2-H3N2 virus with H3N2 specific HA primer; Lane 3-A(2009 H1N1)pdm virus with A(2009 H1N1)pdm specific HA primer; Lane 4-H1N1 virus with H1N1 specific NA primer; Lane 5-H3N2 virus with H3N2 specific NA primer; Lane 6-A(2009 H1N1)pdm virus with A(2009 H1N1)pdm specific NA primer; Lane N-Negative Control (RT-PCR reaction mixture with no RNA).

FIG. 3. Detection of Influenza A positive control viruses by Short-run RT-PCR reaction using 2.0 μl (0.4 μg) RNA. Lane M-100 bp Molecular weight marker; Lane 1 H1N1 virus (208 bp); Lane 2 H3N2 virus (221 bp); and Lane 3 A(2009 H1N1)pdm (200 bp).

FIG. 4A. Detection of Influenza A type H3N2 viruses HA by Short-run RT-PCR assay. Lane L-100 bp Molecular weight marker, Top Lanes: Lane 1-H1N1 positive control (PR8); Lane 2-H3N2 positive control (A/Brisbane/10/2007); Lanes 3-11, wild type H3N2 viruses (see table 2) amplified with H1N1 specific HA primers. Bottom Lanes: Lane 1-H1N1 positive control (PR8); Lane 2-H3N2 positive control (A/Brisbane/10/2007); Lanes 3-11, wild type H3N2 viruses (see table 2) amplified with H3N2 specific HA primers.

FIG. 4B. Detection of Influenza A type H3N2 viruses HA by Short-run RT-PCR assay. Lane L-100 bp Molecular weight marker; Lane 1-H1N1 positive control (PR8); Lane 2-H3N2 positive control (A/Brisbane/10/2007); Lanes 3-14, wild type H3N2 viruses (see table 2) amplified with H3N2 specific HA primers.

FIG. 4C. Detection of Influenza A type H3N2 viruses HA by Short-run RT-PCR assay. Lane L-100 bp Molecular weight marker; Lane 2-H3N2 positive control (A/Brisbane/10/2007); Lanes 3-10, wild type H3N2 viruses (see table 2) amplified with H3N2 specific HA primers.

FIG. 5A. Detection and differentiation of Influenza A type H1N1 viruses NA using subtype specific NA primers. Lane L-100 bp Molecular weight marker; Top Lanes: Lane 1-H1N1 positive control (PR8); Lane 2-H3N2 positive control (A/Brisbane/10/2007); Lanes 3-10, wild type H1N1 viruses (see table 2) amplified with H1N1 specific NA primers. Bottom Lanes Lane 1-H1N1 positive control (PR8); Lane 2-H3N2 positive control (A/Brisbane/10/2007) and Lanes 3-10-wild type H3N2 viruses (see table 2) amplified with H1N1 specific NA primers.

FIG. 5B. Detection of NA of Influenza A type H3N2 viruses with subtype specific NA primers. Lane L-100 bp Molecular weight marker; Top Lanes: Lane 1-H1N1 positive control (PR8); Lane 2-H3N2 positive control (A/Brisbane/10/2007); Lanes 3-10, wild type H3N2 viruses (see table 2) amplified with H1N1 specific NA primers. Bottom Lanes: Lane 1-H1N1 positive control (PR8); Lane 2-H3N2 positive control (A/Brisbane/10/2007); Lanes 3-10-wild type H3N2 viruses (see table 2) amplified with H3N2 specific NA primers.

FIG. 5C. Detection of NA of Influenza A type H3N2 viruses with subtype specific NA primers Lane L-100 bp Molecular weight marker; Top Lanes: Lane 1-H1N1 positive control (PR8); Lane 2-H3N2 positive control (A/Brisbane/10/2007); Lanes 3-14, wild type H3N2 viruses (see table 2) amplified with H1N1 specific NA primers. Bottom Lanes: Lane 1-H1N1 positive control (PR8); Lane 2-H3N2 positive control (A/Brisbane/10/2007); Lanes 3-14-wild type H3N2 viruses (see table 2) amplified with H3N2 specific NA primers.

FIG. 6A. Stability of RT-PCR Mixture (without RNA) stored at different temperatures by RT-PCR amplification of H1N1 HA on day 1. Lane L-100 bp Molecular weight marker. Lane-1 at −20° C.; Lane-2 at 4° C. and Lane-3 at room temperature

FIG. 6B. Stability of the RT-PCR Mixture (without RNA) stored at different temperatures by RT-PCR amplification of H1N1 HA on day 2. Lane L-100 bp Molecular weight marker. Lane-1 at −20° C.; Lane-2 at 4° C. and Lane-3 at room temperature

FIG. 6C. Stability of the RT-PCR Mixture (without RNA) stored at different temperatures by RT-PCR amplification of H1N1 HA on day 50. Lane L-100 bp Molecular weight marker. Lane-1 at −20° C. and Lane-2 at 4° C.

FIG. 6D. Stability of the RT-PCR Mixture (without RNA) stored at different temperatures by RT-PCR amplification of H1N1 HA on day 63. Lane L-100 bp Molecular weight marker. Lane-1 at −20° C. and Lane-2 at 4° C.

FIG. 7A. Gel picture showing some of the reassortant viruses developed in our lab showing mixture of NA types detected by Short-run RT-PCR assay. Lane L-100 bp Molecular weight marker, Lane 1-H1N1 positive control; Lane 2-H3N2 positive control. Lanes 11 and 13 are mixture of N1 and N2 NA types

FIG. 7B. Gel picture showing some of the reassortant viruses developed in our lab showing mixture of NA types detected by Short-run RT-PCR assay. Lane L-100 bp Molecular weight marker, Lane 1-H1N1 positive control; Lane 2-H3N2 positive control. Lane 6 is a mixture of N1 and N2 NA types.

FIG. 7C. Identification of the origin of HA of influenza A H3N2 virus candidate seed viruses by Short-run RT-PCR assay using subtype specific HA primers. Top Lanes: Lane L-100 bp Molecular weight marker; Lane 1-seasonal H1N1 positive virus control (PR8); Lane 2-seasonal H3N2 positive virus control; Lane 3-9-NYMC X-183, NYMC X-185, NYMC X-185xp, NYMC X-187, NYMC X-187A, NYMC X-189 and NYMC X-191 amplified with H1N1 HA primer, respectively. Bottom Lanes: Lane L-100 bp Molecular weight marker; Lane 1-seasonal H1N1 positive virus control (PR8); Lane 2-seasonal H3N2 positive virus control; Lane 3-9-NYMC X-183, NYMC X-185, NYMC X-185xp, NYMC X-187, NYMC X-187A, NYMC X-189 and NYMC X-191 amplified with H3N2 HA primer, respectively.

FIG. 7D. Identification of the origin of NA of influenza A H3N2 virus candidate seed viruses by Short-run RT-PCR assay using subtype specific NA primers. Top Lanes: Lane L-100 bp Molecular weight marker; Lane 1-seasonal H1N1 positive virus control (PR8); Lane 2-seasonal H3N2 positive virus control; Lane 3-9-NYMC X-183, NYMC X-185, NYMC X-185xp, NYMC X-187, NYMC X-187A, NYMC X-189 and NYMC X-191 amplified with H1N1 NA primer, respectively. Bottom Lanes: Lane L-100 bp Molecular weight marker; Lane 1-seasonal H1N1 positive virus control (PR8); Lane 2-seasonal H3N2 positive virus control; Lane 3-9-NYMC X-183, NYMC X-185, NYMC X-185xp, NYMC X-187, NYMC X-187A, NYMC X-189 and NYMC X-191 amplified with H3N2 NA primer, respectively.

FIG. 8. Identification of the origin of HA of influenza A(2009 H1N1)pdm high yield reassortant candidate seed viruses by Short-run RT-PCR assay using subtype specific HA primers. Top Lanes: Lane L-100 bp Molecular weight marker; Lane 1-seasonal H1N1 positive virus control (PR8); Lane 2-A(2009 H1N1)pdm positive virus control; Lane 3-9-NYMC X-181, NYMC X-181A and NYMC X-181B amplified with A(2009 H1N1)pdm HA primer, respectively. Bottom Lanes: Lane L-100 bp Molecular weight marker; Lane 1-seasonal H1N1 positive virus control (PR8); Lane 2-A(2009 H1N1)pdm positive virus control; Lane 3-9-NYMC X-183, NYMC X-185, NYMC X-185xp, NYMC X-187, NYMC X-187A, NYMC X-189 and NYMC X-191 amplified with H1N1 HA primer, respectively.

FIG. 9. Amplification of H3N2 type viruses with H3N2 specific HA primers (550 C/15 min-Reverse-transcription reaction time). Lane L: 100 bp Molecular weight marker; Lanes 1-13 (top row) and Lanes 14-26 (bottom row) are H3N2 viruses (see table 2) amplified at 550 C/15 min. reverse-transcription reaction time.

FIG. 10. Amplification of H1N1 type viruses with H1N1 type NA primers (55° C./15 min-Reverse-transcription reaction time). Lane L: 100 bp Molecular weight marker; Lanes 1-5 are H1N1 viruses (see table 2) amplified at 55° C./15 min. reverse-transcription reaction time.

FIG. 11. Amplification of H3N2 type reassortants (cross between H1N1 and H3N2 viruses) with H1N1 and H3N2 type HA and NA primers. Lane L: 100 bp Molecular weight marker; Top Lanes 1-5-H3N2 reassortants amplified with H1N1 specific HA primer; Top Lanes 6-10-H3N2 reassortants amplified with H1N1 specific NA primer; Bottom Lanes 1-5-H3N2 reassortants amplified with H3N2 specific HA primer; Bottom Lanes 6-10-H3N2 reassortants amplified with H3N2 specific NA primer.

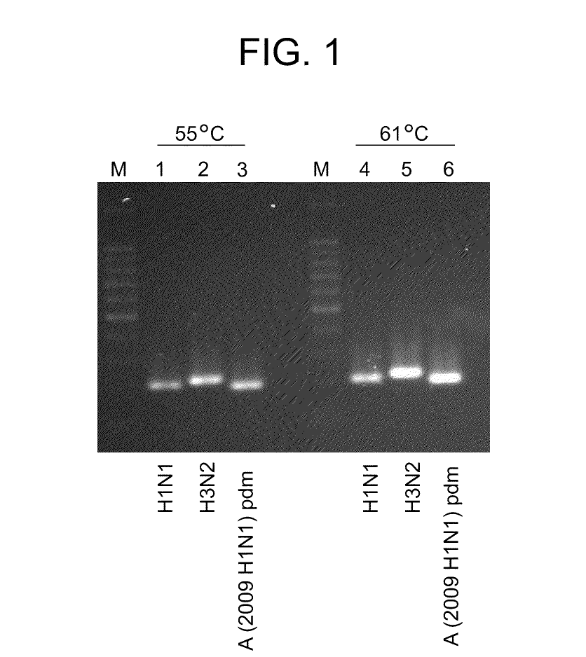

### DETAILED DESCRIPTION

All references cited herein are hereby incorporated herein by reference; in case of any inconsistency the instant disclosure governs.

Molecular biology terms, methods and techniques disclosed herein, unless specifically defined, are used in a manner consistent with their common usage in the field, for example, as described in a textbook, e.g., Sambrook et al., (ed.), Molecular Cloning: A Laboratory Manual. 3^{rd} ed., vol. 1-3, Cold Spring Harbor Laboratory Press, Cold Spring Harbor, N.Y., 2001.

The invention relates to isolated and/or purified nucleic acids e.g., oligonucleotide(s)/polynucleotide(s), the term “isolated and/or purified” includes synthetically prepared nucleic acids, compositions comprising said nucleic acid(s), for rapid detection, identification, diagnosis, and differentiation of certain influenza virus A subtypes, e.g., H1N1, H3N2 and A(2009 H1N1)pdm and method(s) thereof, e.g., nucleic acid-based method(s). The invention further relates to an article of manufacture, e.g., assay, test and/or diagnostic kit, comprising the composition(s) for use in the method(s) described herein, and a method of manufacturing and using said article.

### Oligonucleotides:

In certain embodiments, the invention relates to isolated and/or purified nucleic acids, including synthetically prepared nucleic acids, provided for e.g., as oligonucleotides for use as probes and/or primers for detecting Influenza virus type A. The oligonucleotides of the present inventions are designed to provide recognition of specific sequences in HA and NA genes of influenza virus A subtypes H1N1, H3N2 and A(2009 H1N1)pdm. A person of skill in the art will appreciate that primers and probes are used as single stranded nucleic acids. Also within the scope of the invention are amplified product(s)/amplicon(s) obtained by amplification using said oligonucleotides. Examples of such amplified product(s)/amplicon(s) are shown on Table 4. For example, an amplicon of the HA gene of Influenza virus A subtype seasonal H1N1 can be obtained using an oligonucleotide of SEQ ID NO:1 (forward primer), and an oligonucleotide of SEQ ID NO:2 (reverse primer) in a PCR reaction (Table 1).

The length of the oligonucleotides may range from about 17 nucleotides to about 40 nucleotides, or they may range from 20 to 32 nucleotides in length, or they may range from 23-30 nucleotides in length. Said oligonucleotides may be selected from, but are not limited to, sequences disclosed herein, for e.g., SEQ ID NO:1-SEQ ID NO:12, listed in Table 1. The oligonucleotides of the invention may also include those of SEQ ID NO:1-SEQ ID NO:12 having mutations in 1 or 2 nucleotides.

Based on their property to recognize, and hybridize specifically to complementary/target nucleic acid sequences of interest (from Influenza A virus), a person of skill in the art will appreciate that the nucleic acids (e.g., oligonucleotides) of the present invention may be used as probes and/or primers for detection, identification, diagnosis, and differentiation of Influenza virus A subtypes H1N1, H3N2 and A(2009 H1N1)pdm in a sample, e.g., biological sample. Said nucleic acids may be labeled with suitable labels, and/or tags, and/or reporter molecules. Examples of such labels are biotin, avidin and/or streptavidin, fluorescent label, digoxygenin, radiolabel, etc.

### RT-PCR

In certain embodiments, the invention relates to a method, test, or assay (e.g., RT-PCR) comprising oligonucleotides of the invention, provided as primers, which may be selected from, but are not limited to, sequences disclosed as SEQ ID NO:1-SEQ ID NO:12 on Table 1 of the present disclosure for detection, identification, diagnosis, and differentiation of Influenza virus A subtypes H1N1, H3N2 and A(2009 H1N1)pdm in a sample, e.g., biological sample. It can be readily appreciated by a person of skill in the art that appropriate oligonucleotide pairs, consisting of a forward primer and a reverse primer, selected from SEQ ID NO:1-SEQ ID NO:12 are used for the PCR amplification reactions described herein. A primer pair may also be described as consisting of a given oligonucleotide and a counterpart oligonucleotide, e.g., SEQ ID NO:1 is a counterpart of SEQ ID NO:2, and vice versa.

In another embodiment, the invention relates to an RT-PCR assay comprising oligonucleotides, which may be selected from, but are not limited to, sequences disclosed as SEQ ID NO:1-SEQ ID NO:4 on Table 1 of the present disclosure to detect, identify, diagnose and distinguish influenza virus A subtype seasonal H1N1.

In another embodiment, the invention relates to an RT-PCR assay comprising oligonucleotides, which may be selected from, but are not limited to, sequences disclosed as SEQ ID NO:5-SEQ ID NO:8 on Table 1 of the present disclosure to detect, identify, diagnose and distinguish influenza virus A subtype seasonal H3N2.

In another embodiment the invention relates to an RT-PCR assay comprising oligonucleotides, which may be selected from, but are not limited to, sequences disclosed as SEQ ID NO:9-SEQ ID NO:12 on Table 1 of the present disclosure to detect, identify, diagnose and distinguish influenza virus A subtype A(2009 H1N1)pdm.

In another embodiment, the invention relates to a Short-run RT-PCR assay comprising oligonucleotides, which may be selected from, but not limited to, sequences disclosed (on Table 1 of the present disclosure) as SEQ ID NO:1-SEQ ID NO:4 to detect, identify, diagnose and distinguish influenza virus A subtype seasonal H1N1; SEQ ID NO:5-SEQ ID NO:8 to detect, identify, diagnose and distinguish influenza virus A subtype seasonal H3N2; and SEQ ID NO:9-SEQ ID NO:12 to disclosure to detect, identify, diagnose and distinguish influenza virus A subtype A(2009 H1N1)pdm, wherein the RT and PCR reactions are performed using a combined and/or single reaction mixture (i.e., one step RT-PCR). While the Short-run RT-PCR method(s) described herein are particularly useful for qualitative detection (of the specific Influenza A virus subtypes), a person of skill in the art will appreciate that quantitative analyses (i.e., to determine quantity of target sequence(s) in a sample(s)) can also be readily performed, by such individual, when necessary.

In further embodiments, the invention relates to real time RT-PCR method(s)/assay(s)/test(s), and multiplex RT-PCR method(s)/assay(s)/test(s). When used in a multiplex assay, multiple primer pairs selected from, but not limited to, SEQ ID NO:1 to SEQ ID NO:12 may be simultaneously used in the reaction. For example, SEQ ID. NO: 1 and SEQ ID NO:2 (directed to HA gene of seasonal H1N1) and SEQ ID NO: 5 and SEQ ID NO: 6 (directed to HA gene of seasonal H3N2) may be used in combination. It can be appreciated that other primer pair combinations selected from, but not limited to, the oligonucleotides of the invention can be used.

General method(s) and techniques for performing RT-PCR are well-documented in Molecular Biology protocols, and can be readily found in a textbook (e.g., Sambrook and Russell, 2001). Chemicals and reagents for performing the methods are readily available from commercial sources. A person of skill in the art will appreciate that such chemicals and reagents may be purchased individually or can be purchased as kits from commercial sources.

Methods for isolating Influenza viral RNA from a virus sample or a sample (e.g., biological sample) from virus-containing material (e.g. laboratory, clinical), or from a subject in need thereof, are well-known to persons of skill in the field.

In the RT-PCR (of the invention), the steps of Reverse transcription (RT) and subsequent polymerase chain reaction (PCR), can be performed in two separate reaction mixtures or in a single reaction mixture (i.e., one-step). The method can be practiced using chemicals and reagents, purchased individually or as kit(s), from commercial sources.

The RT-PCR of the invention can be performed in a short period of time. Starting from viral RNA to obtaining final results, including confirmatory analysis, the method requires, e.g., 90 minutes or less, e.g., about 30 minutes to about 90 minutes, e.g., from about 30 minutes to either about 60, about 70 or about 80 minutes, e.g., from about 45 minutes to either about 60, about 70, about 75, about 80, or about 90 minutes. In certain embodiments, final confirmatory analysis/results can be completed/obtained in about 75 minutes by carrying out the step of reverse transcription (RT) in about 15 minutes, the step of PCR amplification in about 30 minutes, and gel electrophoresis and visualization in about 30 minutes. As used herein, “confirmatory analysis” refers to obtaining final results, including analyzing the amplified product (amplicon) e.g., by visualization after gel electrophoresis. In the context of point-of-care, a person of skill in the art will appreciate that confirmation of amplified product will be by visualization of appropriate bands on an agarose gel following electrophoresis. However, a person of skill in the art can envision other methods to confirm the presence or absence of amplified product.

As used herein, “biological sample” refers to a sample (or specimen) of any material (e.g., swab, fluid or tissue) obtained from a human, avian, or animal, and includes laboratory specimens (e.g., isolated virus, reference virus standards, cell/tissue culture fluids), clinical specimens (including those obtained post-mortem) and prophylactic preparations (e.g., vaccine, seed virus for vaccine). Biological material isolated and/or purified from a biological sample are also within the scope of the definition. Also within the scope of this definition are samples which are freshly-obtained and/or prepared or stored (e.g., at 4° C. or frozen). Examples of samples include, but are not limited to, nasopharyngeal exudates and/or swabs, throat swabs, tracheal swab, saliva, urine, blood, serum, plasma, lung tissue, tracheal tissue, avian cloacal samples or swabs, etc.

As used herein, “detection or detecting” refers to determining the presence or absence (e.g., qualitatively) of an Influenza A virus subtype e.g., H1N1, H3N2 and A(2009 H1N1)pdm by the method(s) disclosed herein.

As used herein, “sensitivity” refers to the percentage of “true influenza cases” detected as positive by the method(s)/assay/(s)test(s) disclosed herein. It should be noted that this is not the same as the term “analytical” sensitivity of the method(s)/assay(s)/test(s)), the use of which is consistent with its accepted use in the field.

As used herein, “specificity” is the percentage of “true non-influenza cases” detected as being negative by the method(s)/assay/(s)test(s) disclosed herein.

As used herein, “subject in need thereof” refers to humans or animals (including avians, e.g., poultry, and swine, e.g., pigs) presenting with symptoms that meet the surveillance case definition of an influenza-like illness (as provided by World Health Organization and/or U.S. Centers for Disease Control and Prevention) and/or with flu-like or flu-associated symptoms that can be readily recognized by a medical practitioner in the field. Also within the scope of this definition are hospitalized patients for whom influenza infection is clinically suspected despite a negative result on a rapid influenza diagnostic test; subjects whose deaths are believed to be influenza-associated; subjects at risk of developing influenza infection, e.g., immunocompromised subjects (e.g., subjects on steroid therapy); subjects with HIV/AIDS; chronically ill subjects; subjects undergoing cancer treatments (e.g., chemotherapy, radiation therapy, hormone therapy); infants and young children; senior individuals (e.g., >age 60-65); subjects for whom a diagnosis of influenza will inform decisions regarding clinical care, infection control, or management of close contacts.

### Kits

In another embodiment, the invention relates to a packaged article(s), e.g., an article of manufacture, such as an assay and/or diagnostic kit, comprising the composition(s) of the invention, optionally with a label(s) and/or with instructions for use. Such label(s) include(s) ingredients, amounts or dosages, and/or indications. Such instructions include directing or promoting, including advertising, use of said article of manufacture. Such instructions may be provided for example an illustrative information (e.g., drawing and/or text) provided by the manufacture.

In particular, the present invention provides kit(s) for simple, rapid, Short-run RT-PCR test/assay to detect, identify and distinguish Influenza A virus subtypes, particularly seasonal H1N1, seasonal H3N2, A(2009 H1N1)pdm. Such kit(s) may comprise one or more nucleic acids (e.g., primers and/or probes) of the invention, for example a kit may contain primers consisting of one or more oligonucleotides of the invention, e.g., SEQ ID NO: 1, SEQ ID NO: 2, SEQ ID NO:3, SEQ ID NO:4 for detecting, identifying and diagnosing Influenza virus subtype H1N1(seasonal). The kit may also comprise appropriate primer pairs for detecting and distinguishing Influenza virus A subtypes disclosed herein (seasonal H1N1, seasonal H3N2, A(2009 H1N1)pdm) in a single test reaction. The kit according to the invention may optionally include a positive control nucleic acid, for example a nucleic acid, or at least a portion thereof, comprising regions from HA and NA genes of Influenza A virus subtypes disclosed herein, as either RNA (viral) or DNA. A person of skill in the art can appreciate that reagents, including oligonucleotides in the kit may be provided in individual, separate containers or pre-mixed (e.g., master mix) in single or multiple containers.

In certain embodiments, the kit of the invention is designed for simultaneous detection of all three Influenza virus A subtypes (seasonal H1N1, seasonal H3N2, A(2009 H1N1)pdm), i.e., the kit comprises at least three sets of primer pairs, and up to six sets of primer pairs, i.e., at least one primer pair per Influenza virus A subtype. A person of skill in the art will appreciate that suitable primer pairs can be selected from SEQ ID NO:1-SEQ ID NO:12.

In other embodiments, the kit of the invention may contain one or two pair(s) of primers specific for one Influenza A virus subtype, e.g., SEQ ID NO:1 and SEQ ID NO:2, or SEQ ID NO:1, SEQ ID NO:2, SEQ ID NO:3, and SEQ ID NO:4, to detect Influenza A virus subtype seasonal H1N1.

In another embodiment, the invention relates to a method of manufacturing an article of manufacture (e.g. an assay and/or diagnostic kit) comprising any of the compositions of the invention described herein, packaging the composition(s), as one or more packages, to obtain an article of manufacture and instructing, directing or promoting the use of the article of manufacture for any of the uses described herein. Such instructing, directing or promoting includes advertising.

The invention will be more readily understood through reference to the following examples which are provided by way of illustration, and is not intended to be limiting of the present invention.

### EXAMPLES

### Example 1

### Detection of Influenza A Virus Subtypes H1N1, H3N2, A(2009 H1N1)pdm by a Rapid, Short-Run RT-PCR

### Method:

### Primer Design:

To determine the conserved regions, the sequence of HA and NA genes of Influenza A subtypes H1N1, H3N2, A(2009 H1N1)pdm viruses were retrieved from the publicly-accessible Genbank database maintained by the National Center for Biotechnology Information (NCBI) (www.ncbi.nlm.nih.gov). To identify conserved regions within and between subtypes, the CLUSTALW algorithm (Thompson et al., 1994. CLUSTAL W: improving the sensitivity of progressive multiple sequence alignment through sequence weighting, position-specific gap penalties and weight matrix choice. Nucleic Acids Res., 22(2), 4673-4780) was used to generate a multiple sequence alignment of identical viruses obtained from NCBI. From the aligned sequences, conserved regions which are highly subtype specific and show no sequence alignment with other subtypes were selected; no reactivity among H1N1, H3N2 and A(2009 H1N1)pdm was seen. Two primer sets/pairs (for each of the tested influenza virus subtypes) were designed for the selected regions using PrimerDesign Program and were synthesized by Integrated DNA Technologies (Coralville, Iowa); oligonucleotide sequences for the primers which gave the desired results (e.g., specificity) in the present RT-PCR assay (discussed below) are shown on Table 1.

TABLE 1
Primers used for Shortrun RT-PCR detection and differentiation of
Influenza A viruses

| [SEQ ID NO:] Primer name | | Primer sequence | Product size (bp) |
|---|---|---|---|
| Seasonal H1N1 virus | | | |
| [SEQ ID NO: 1] | HA F | AAAGAAAGCTCATGGCCCAACCAC | |
| [SEQ ID NO: 2] | HA R | GTTGTTCCTTACTGTTAGACGGGTG | 208 (438-645) |
| [SEQ ID NO: 3] | NA F | AATAACCATTGGATCAATCTGTCTGGT | |
| [SEQ ID NO: 4] | NA R | AATTGCCGGTTAATATCACTGAAGTTGTG | 200 (41-240) |
| Seasonal H3N2 virus | | | |
| [SEQ ID NO: 5] | HA F | ACTTCCCGGAAATGACAACAGCAC | |
| [SEQ ID NO: 6] | HA R | ACTGAGGGTCTCCCAATAGAGCAT | 221 (63-283) |
| [SEQ ID NO: 7] | NA F | ACGATTGGCTCTGTTTCTCTCACC | |
| [SEQ ID NO: 8] | NA R | CCTTCTCTATGGTGGTGTTGGTCA | 202 (25-226) |
| A(2009 H1N1)pdm virus | | | |
| [SEQ ID NO: 9] | HA F | ACAAAGGTGTAACGGCAGCATGTC | |
| [SEQ ID NO: 10] | HA R | TGCATTCTGATAGAGACTTTGTTGGTCAGC | 200 (437-636) |
| [SEQ ID NO: 11] | NA F | ACCATTGGTTCGGTCTGTATGAC | |
| [SEQ ID NO: 12] | NA R | CAGCAGCAAAGTTGGTGTTGCTGA | 205 (25-229) |

F: Forward primer. R: Reverse primer.

### Viral RNA Extraction and RT-PCR Assay:

Viral RNA was extracted from the infected allantoic fluid harvested from embryonated chicken eggs using the QIAamp Viral RNA extraction kit (Qiagen Cat #52904, Valencia, Calif.) as per the manufacturer's recommendations. The RNA samples were stored at −20° C. until further use. The HA and NA genes were amplified by Short run RT-PCR using a one step RT-PCR kit (Clontech #RR024A, Mountain View, Calif.). The reaction was carried out according to the manufacturer's instructions in 0.2 ml Eppendorf tubes containing 2.0 μl or 0.4 μg of viral RNA, 1 μl of 10× One Step RNA PCR Buffer, 1 μl of 5 mM MgCl2, 2 μl of 1 mM dNTP, 0.5 μl of 0.8 U RNase Inhibitor, 0.5 μl of 0.1 U AMV RTase XL, 0.5 μl of 0.1 U AMV-Optimized Taq, 0.5 μl of (10 pmoles/μl) each of forward and reverse primers, and RNase free H₂O up to a total volume of 10 μl. Cycling conditions were as follows: 30 min at 15° C. (reverse-transcription), 1 min at 94° C. (initial PCR activation), followed by 15 cycles of 20 s at 94° C., 30 s at 55° C., and 30 s at 70° C. and then final extension for 5 min at 72° C. The positive controls and negative controls (RT-PCR mix+H2O) were included appropriately. The reactions were performed on an Eppendorf Mastercycler®. The amplified products were visualized on a 2% agarose gel with added ethidium bromide (10 mg/ml) in Tris-acetate EDTA buffer (Sambrook and Russell, 2001). A 100 bp DNA ladder (Promega, Madison, Wis.) was used as a molecular weight marker. The PCR samples were stored at 4° C. until used.

### Initial Standardization of RT-PCR Assay:

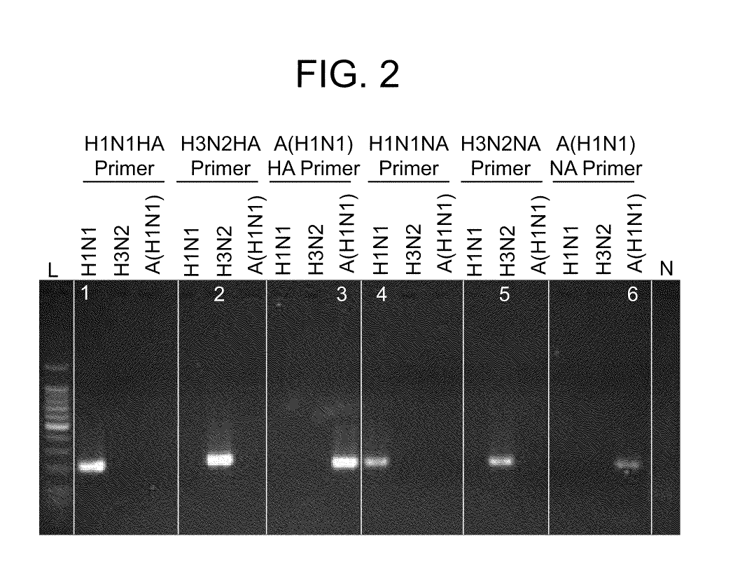

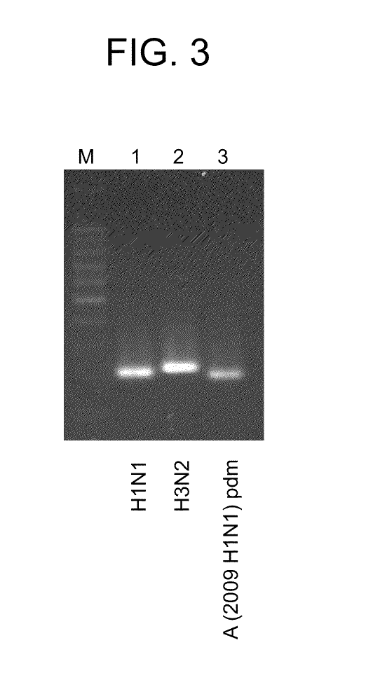

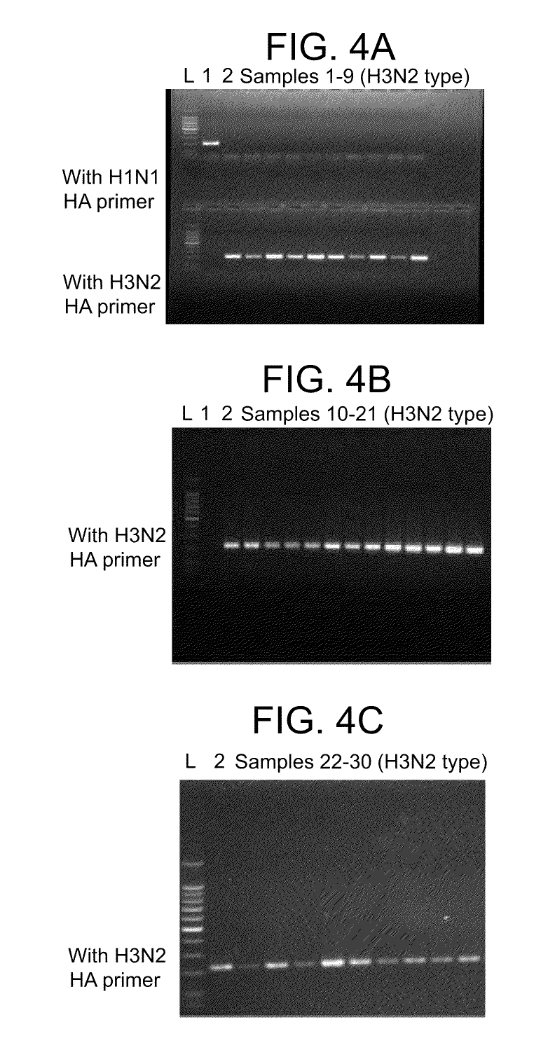

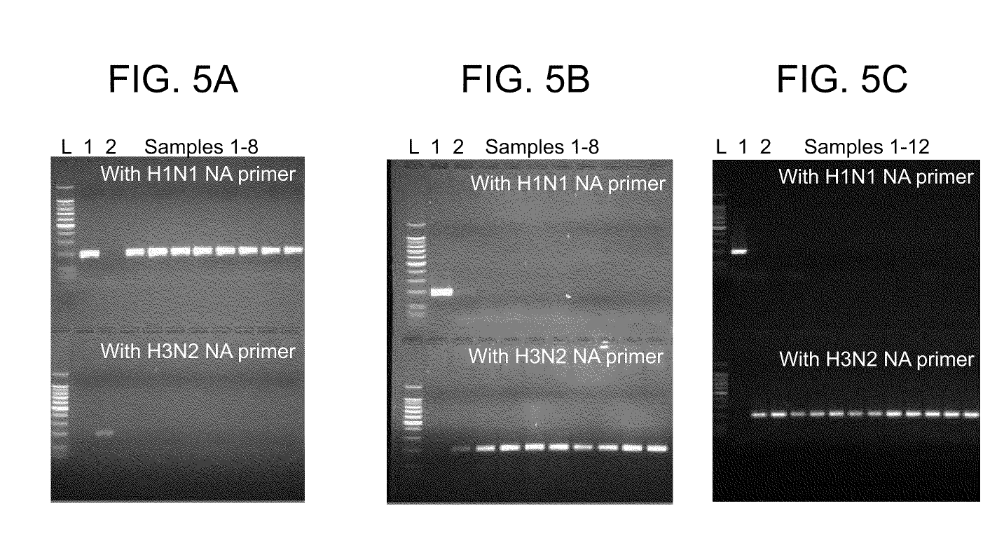

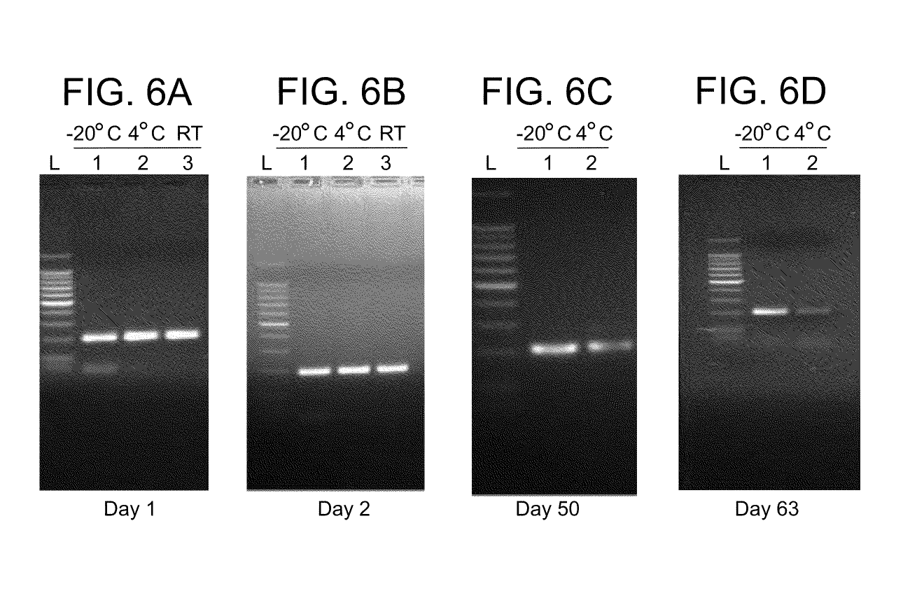

Influenza A A/Puerto Rico/8/34 (PR8) (H1N1), A/Brisbane/10/2007 (H3N2) and A/California/07/2009 [A(2009 H1N1)pdm] viruses were used as positive controls. Initially, the developed assay was standardized for amplification parameters using the positive control viruses. The HA and NA genes were amplified for 30 seconds at 55° C. and 61° C. annealing temperatures (Tₘ), using the aforementioned reaction conditions. Since there was no difference in amplification efficiency at different annealing temperatures (data not shown), all further experiments were carried out at 55° C. Tₘ. The analytical sensitivity of the assay was determined by using different concentrations of RNA (approximately 0.4 μg to 2 μg per reaction) and in all cases consistent amplification was detected with lower limiting volumes of 2 μl (0.4 μg) of RNA. The positive control viruses were amplified with their respective subtype specific primers and showed no cross-amplification with other subtypes, demonstrating the specificity of the assay (FIG. 2).

### Confirmation of Amplified Positive Control Products:

Gel Electrophoresis was performed for 30 minutes at 100V current. Further, commercially prepared gels can be used to save preparation time (however, gels are prepared while the reaction is running).

To confirm the identities of the amplified products, the resultant DNA bands from the positive control viruses were excised and purified using QIAquick gel extraction kit (Qiagen, #28704, Valencia, Calif.). Purified DNA fragments were sequenced commercially (McLab, South San Francisco, Calif.). The raw sequence data was edited using EditSeq® and the nucleotide sequence homology was determined using MegAlign® module in the LASERGENE package (DNASTAR Inc., Madison, Wis.). Alignment confirmed accuracy as each distinct band was identified as the appropriate viral genome segment.

### BLAST Results:

Both the Primer sequence and the amplified product sequences were ‘blasted’ at the NCBI site (http://www.ncbi.nlm.nih.gov/sites/BLAST). The results showed Influenza A virus subtype specificity. The high specificity of the sequences was shown by the presence of these sequences in the genes of the recent outbreak viruses from the GenBank database.

### Viruses Tested in the Assay:

Further, the Short run RT-PCR assay was validated using influenza A viruses belonging to different subtypes available in our laboratory (Table 2). The panel of viruses used for validating the assay was received as egg-adapted viruses from the U.S. Centers for Disease Control, Georgia.

The H1N1 viruses included in this assay were A/Puerto Rico/8/1934 [Genbank accession # HA (CY033577) and NA (CY033579)], A/USSR/90/1977, A/Brazil/11/1978, A/Chile/1/1983, A/Texas/36/1991, A/Beijing/262/1995, A/Shenzhen/227/1995, A/New Calcdonia/20/1999, A/St. Petersburg/8/2006, A/South Dakota/06/2007, and A/Hong Kong/1870/2008.

The H3N2 viruses included in this assay were A/Aichi/2/1968, A/England/42/1972, A/Port Chalmers/1/1973, A/Victoria/3/1975, A/Bangkok/1/1979, A/Leningrad/360/1986, A/Sichuan/2/1987, A/Shanghai/11/1987, A/Beijing/32/1992, A/Harbin/15/1992, A/Shangdong/9/1993, A/Johannesburg/33/1994, A/Moscow/10/1999, A/Panama/2007/1999, A/California/32/1999, A/Ulan Ude/01/2000, A/Wyoming/03/2003, A/Texas/40/2003, A/Fujian/445/2003, A/New York/55/2004, A/Mississippi/05/2004, A/Wellington/01/2004, A/Wisconsin/67/2005, A/Nepal/921/2006, A/Brisbane/09/2006, A/Wisconsin/03/2007, A/Brisbane/10/2007 [Genbank accession # HA (CY035022) and NA (CY035024)], A/Uruguay/716/2007, A/Wisconsin/15/2009 and A/Guangdong-Luohu/1256/2009.

A(2009 H1N1)pdm included in this assay were A/California/07/2009 [Genbank accession # HA (FJ981613) and NA (FJ984386)], A/New York/18/2009, A/Mexico/4108/2009 and 1976 swine influenza H1N1 virus, A/New Jersey/11/1976.

TABLE 2
Virus Samples tested in the Short run RT-PCR assay with
respective subtype specific primers

| Viruses | HA primer | NA primer |
|---|---|---|
| H1N1 | H1N1 | H1N1 |
| A/Puerto Rico/8/1934 | + | + |
| A/Texas/36/1991 | + | + |
| A/Beijing/262/1995 | + | + |
| A/Shenzhen/227/1995 | −− | −− |
| A/New Caledonia/20/1999 | + | + |
| A/St.Petersburg/8/2006 | + | + |
| A/South Dakota/06/2007 | + | + |
| A/Hong Kong/1870/2008 | + | + |
| A/USSR/90/1977 | + | + |
| A/Brazil/11/1978 | + | + |
| A/Chile/1/1983 | + | + |
| H3N2 | H3N2 | H3N2 |
| A/Aichi/2/1968 | + | + |
| A/England/42/1972 | + | + |
| A/PortChalmers/1/1973 | + | + |
| A/Victoria/3/1975 | + | + |
| A/Bangkok/1/1979 | + | + |
| A/Leningrad/360/1986 | + | + |
| A/Sichuan/2/1987 | + | + |
| A/Shanghai/11/1987 | + | + |
| A/Beijing/32/1992 | + | + |
| A/Harbin/15/1992 | + | + |
| A/Shangdong/9/1993 | + | + |
| A/Johannesburg/33/1994 | + | + |
| A/Moscow/10/1999 | + | + |
| A/Panama/2007/1999 | + | + |
| A/Ulan Ude/01/2000 | + | + |
| A/California/32/1999 | + | + |
| A/Wyoming/03/2003 | + | + |
| A/Fujian/445/2003 | + | + |
| A/Texas/40/2003 | + | + |
| A/Wellington/01/2004 | + | + |
| A/New York/55/2004 | + | + |
| A/Mississippi/05/2004 | + | + |
| A/Wisconsin/67/2005 | + | + |
| A/Nepal/921/2006 | + | + |
| A/Wisconsin/03/2007 | + | + |
| A/Brisbane/09/2006 | + | + |
| A/Brisbane/10/2007 | + | + |
| A/Uruguay/716/2007 | + | + |
| A/Wisconsin/15/2009 | + | + |
| A/GL/1256/2009 | ± | + |
| A(2009 H1N1)pdm | A(2009 H1N1)pdm | A(2009 H1N1)pdm |
| A/California/07/2009 | + | + |
| A/New York/18/2009 | + | + |
| A/Mexico/4108/2009 | + | + |
| A/New Jersey/11/1976 | + | − |

### Sensitivity and Specificity of the Assay:

Sensitivity is the percentage of “true influenza cases” detected as positive by a test. Specificity is the percentage of “true non-influenza cases” detected as being negative by a test. The amplification data show high specificity of the assay for the influenza viral genes (Table 3).

TABLE 3
Sensitivity and Specificity of the Short run RT-PCR assay

| | H1N1 | | H3N2 | | A(2009 H1N1)pdm | |
|---|---|---|---|---|---|---|
| | primers | | primers | | primer | |
| Virus Subtype | HA | NA | HA | NA | HA | NA |
| H1N1 | 91.6^{a} | 91.6^{a} | 0^{#} | 0^{#} | 0^{#} | 0^{#} |
| H3N2 | 0^{#} | 0^{#} | 100^{a} | 100^{a} | 0^{#} | 0^{#} |
| A(2009 H1N1)pdm | 0^{#} | 0^{#} | 0^{#} | 0^{#} | 100^{a} | 100^{a} |

^{a}Sensitivity of the assay calculated in terms of percentage (91.6-100%). The NA gene sequence of 1976 swine influenza virus, A/New Jersey/November/1976 is different than influenza virus A(2009 H1N1)pdm NA gene. Hence the NA primer specific for A(2009 H1N1)pdm could not detect the NA of A/New Jersey/November/1976.
^{#}Specificity of the assay calculated in terms of percentage (no false positive seen, i.e. specificity = 100%).
Specificity was determined as follows:
H1N1 viruses with H3N2 and A(2009 H1N1)pdm HA and NA primers
H3N2 viruses with H1N1 and A(2009 H1N1)pdm HA and NA primers
A(2009 H1N1)pdm viruses with H1N1 and H3N2 HA and NA primers

The A(2009 H1N1)pdm was handled per CDC guidelines, and the primers which were used for A(2009 H1N1)pdm are shown in Table 1. The primers of A(2009 H1N1)pdm genes were designed based on A/California/07/2009 virus sequence. The assay amplified HA and NA genes of A(2009 H1N1)pdm at 100% specificity. Further blast analysis by the selected A(2009 H1N1)pdm amplified region at NCBI demonstrated 100% sequence homology for HA and NA genes in the first 100 blast hits. There was no cross reactivity with either seasonal H1N1 and/or H3N2 viruses.

### Stability of the Reagents (E.G., Ability to Withstand Heat and Cold):

The Short-run RT-PCR reaction master mixture was made from individual reagent tubes and was added with both forward and reverse primers. The mixture was stored at −20° C., 4° C., room temperature (R.T.), and at 37° C. The viral HA genes were amplified from reaction mixtures stored at −20° C., 4° C. and R.T. on day 1 and on day 2. The testing was then repeated at day 10, day 50, and day 63. This experiment showed that the reaction mixture can be stored at 4° C. at least 50 days, and up to 63 days when refrigerated (longer times not tested). Amplification products were detected at all tested time points for reaction mixtures stored at −20° C. and 4° C.

### Rapid Turnaround Time:

Total time to get results: 90 minutes
RT-PCR Reaction time: 60 minutes
Gel Electrophoresis: 30 minutes at 100V current. Commercially prepared gels can be used to save preparation time (however, gels are prepared while RT-PCR reaction is running)

### Advantages Over Other Detection Tests:

Time: Required only 90 minutes to obtain final results
Sensitivity: 91.6-100% with the available virus samples tested (Table 2 and Table 3).
Specificity: 100% (see Table 3, no false positives seen i.e. specificity=100%).
Technical Skills Not labor consuming and requires less laboratory skills
Stability of the reagents: Equipment: Conventional PCR machine and electrophoresis apparatus are sufficient

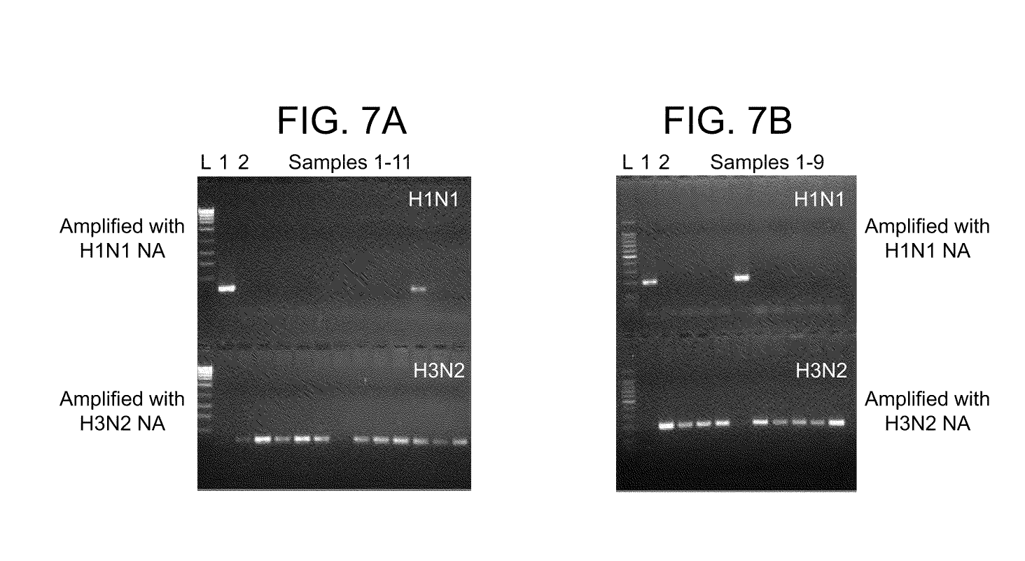

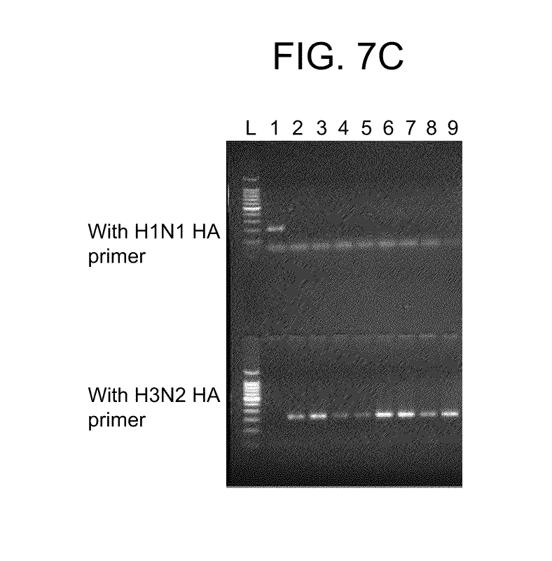

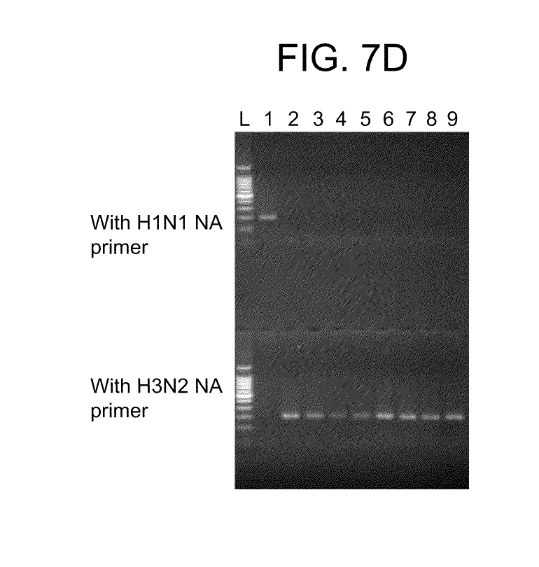

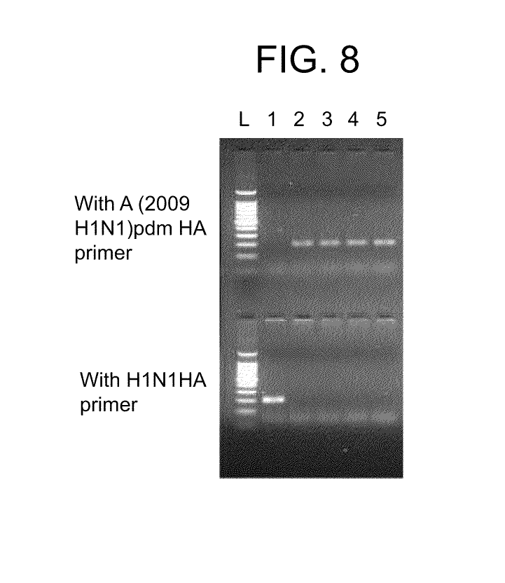

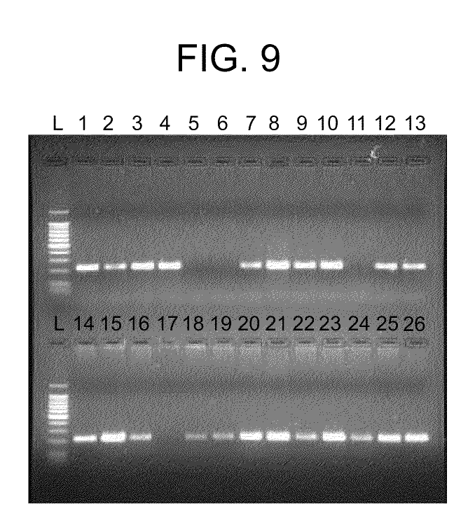

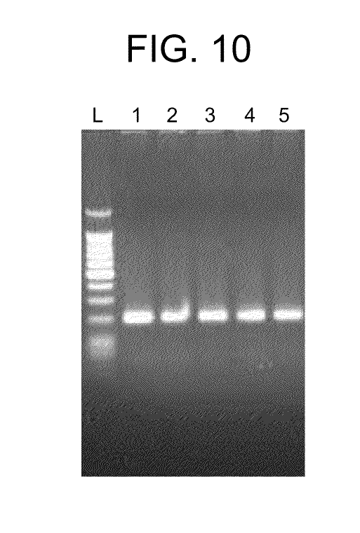

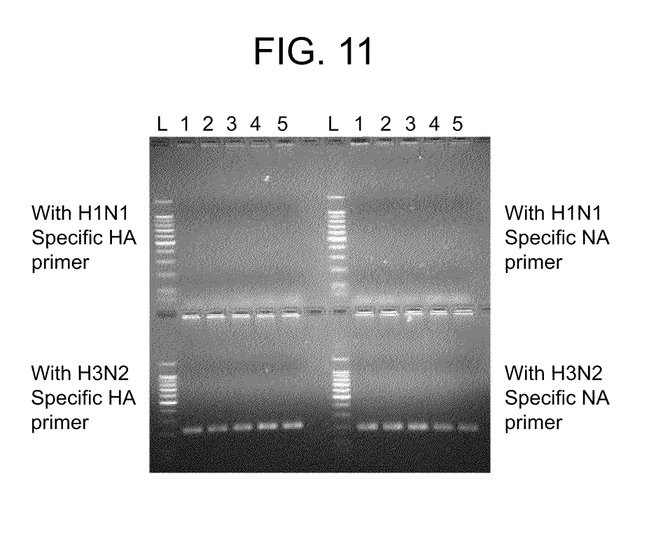

Subsequent experiments have shown that the RT-PCR reaction time was reduced to 45 minutes and the total time to obtain final confirmatory results was reduced to 75 minutes (e.g., FIGS. 7C, 7D and 8).

TABLE 4
Amplicon sequences for the HA and NA genes are given below:
Amplified HA gene Sequence:
[SEQ ID NO: 13] H1N1 (208 bp)
AAAGAAAGCTCATGGCCCAACCACAACACAAACGGAGTAACGGCAGCATGCT
CCCATGAGGGGAAAAGCAGTTTTTACAGAAATTTGCTATGGCTGACGGAGAAG
GAGGGCTCATACCCAAATCTGAAAAATTCTTATGTGAACAAAAAAGGGAAAGA
AGTCCTTGTACTGTGGGGTATTCATCACCCGTCTAACAGTAAGGAACAAC
[SEQ ID NO: 14] H3N2 (221 bp)
ACTTCCCGGAAATGACAACAGCACGGCAACGCTGTGCCTTGGGCACCATGCAG
TACCAAACGGAACGATAGTGAAAACAATCACGAATGACCAAATTGAAGTTACT
AATGCTACTGAGCTGGTTCAGAGTTCCTCAACAGGTGGAATATGCGACAGTCC
TCATCAGATCCTTGATGGAGAAAACTGCACACTAATAGATGCTCTATTGGGAG
ACCCTCAGT
[SEQ ID NO: 15] A(2009 H1N1)pdm (200 bp)
5′ACAAAGGTGTAACGGCAGCATGTCCTCATGCTGGAGCAAAAAGCTTCTACAA
AAATTTAATATGGCTAGTTAAAAAAGGAAATTCATACCCAAAGCTCAGCAAAT
CCTACATTAATGATAAAGGGAAAGAAGTCCTCGTGCTATGGGGCATTCACCAT
CCATCTACTAGTGCTGACCAACAAAGTCTCTATCAGAATGCA3′
Amplified NA gene Sequence:
[SEQ ID NO: 16] H1N1 (200 bp)
AATAACCATTGGATCAATCTGTCTGGTAGTCGGACTAATTAGCCTAATATTGCA
AATAGGGAATATAATCTCAATATGGATTAGCCATTCAATTCAAACTGGAAGTC
AAAACCATACTGGAATATGCAACCAAAACATCATTACCTATAAAAATAGCAC
CTGGGTAAAGGACACAACTTCAGTGATATTAACCGGCAATT
[SEQ ID NO: 17] H3N2 (202 bp)
ACGATTGGCTCTGTTTCTCTCACCATTTCCACAATATGCTTCTTCATGCAAATTG
CCATCTTGATAACTACTGTAACATTGCATTTCAAGCAATATGAATTCAACTCCC
CCCCAAACAACCAAGTGATGCTGTGTGAACCAACAATAATAGAAAGAAACATA
ACAGAGATAGTGTATCTGACCAACACCACCATAGAGAAGG
[SEQ ID NO: 18] A(2009 H1N1)pdm (205 bp)
ACCATTGGTTCGGTCTGTATGACAATTGGAATGGCTAACTTAATATTACAAATT
GGAAACATAATCTCAATATGGATTAGCCACTCAATTCAACTTGGGAATCAAAA
TCAGATTGAAACATGCAATCAAAGCGTCATTACTTATGAAAACAACACTTGGG
TAAATCAGACATATGTTAACATCAGCAACACCAACTTTGCTGCTG

### Example 2

### Short-Run RT-PCR Assay for Identifying the Origin of Parental Genes in Influenza A Virus Vaccine ‘Seed’ Candidate

Generation of High Yielding (hy) Reassortant(s):

Because of the segmented structure of its genome, Influenza viruses have the ability to reassort (i.e., exchange gene segments between two viruses). By taking advantage of this ability (to reassort), high yielding (hy) or high growth reassortants (hgr) can be prepared and used, e.g., as ‘seed’ viruses for the preparation of the virus necessary for production of influenza vaccines. Generation of hy reassortant seed viruses for influenza A vaccine requires incorporation of the two genes for the surface glycoproteins, HA and NA from wild-type (wt) or ‘target’ virus with one to six of the remaining genes from the hy donor virus (PR8). The H3N2 subtype hy reassortants are generated using PR8 as the hy donor and H1N1 hy reassortants are generated using an H3N2 hy donor [e.g., NYMC X-157 (subtype H3N2 hy reassortant with HA and NA genes for wt virus, A/New York/55/2004(H3N2) and 6 internal genes from PR8, developed at New York Medical College, Valhalla, N.Y. (NYMC)] to allow a clear antigenic distinction between H3N2 and H1N1 subtypes for neutralization of any viruses with HA and NA from the hy donor.

Current HA and NA Identification Procedures:

Identification of parental origin of HA and NA genes (preferably from wt/target virus) is carried out to identify the reassortant(s) with correct HA and NA. Currently, the identification of the reassortants with desired HA and NA genes are done based on the Hemagglutination Inhibition assay (takes about 4 hours to get the result), the Neuraminidase Inhibition assay (takes about 16 h to get the result) (serological assays) and RT-PCR/Restriction Fragment Length Polymorphism (RFLP) (takes 24 to 48 hours to get the final confirmatory result).

Development of an H3N2 hy Reassortant(s):

The following steps are typically involved in the development of an H3N2 hy reassortant.

Step 1: amplification of wt/target virus (fresh passage, after receiving from CDC) (42 hrs).

Step 2: Co-infection of wt/target and hy donor (PR8) viruses into 10-12-day-old specific pathogen-free (SPF) eggs (42 hrs).

Steps 3-5: Antibody Selection in order to eliminate progeny viruses containing the HA and NA from the donor virus (42 hrs for each step); Step 3 is repeated two additional times as Step 4 and Step 5 to insure no trace of hy donor HA and NA genes after Step 5).

Step 6: Amplification for an additional passage in eggs (42 hrs).

Step 7-9: Cloning by Limiting Dilution to select the reassortant(s) with the highest HA titer and a gene constellation closest to 6:2 (6 genes from PR8 and the two surface antigens, HA and NA, from wt/target virus) (42 hrs for each step). Step 7 Cloning is repeated as Step 8 and Step 9 to insure that a reassortant seed is produced with a single gene composition.

Step 10: Final Amplification (42 hrs) and shipment of the hy reassortant(s) to CDC and vaccine manufacturers.

Short-Run RT-PCR Technique:

The use of Short-run RT-PCR rapidly identifies the parental origin of HA and NA genes with a reaction time of 75 to 90 minutes, thus expediting the process of identification of candidate ‘seed’ viruses. Use of Short-run RT-PCR permits use of 16-18 hr replication times, thus greatly reduces the overall time to development of hy seed viruses. Based on the results presented in FIGS. 7C, 7D and 8, with the use of this process, it is expected that the time for ‘seed’ virus identification can be reduced to approximately 10 days.

Reassortants Checked with the Developed Assay:

We have applied this assay in generating 10 hy reassortants starting from NYMC X-181, NYMC X-181A and NYMC X-181B (A(2009 H1N1)pdm hy reassortants), NYMC X-183, NYMC X-185, NYMC X-185xp, NYMC X-187, NYMC X-187A, NYMC X-189, and NYMC X-191 (subtype H3N2 hy reassortants).

NYMC X-181 was used for production of A 2009 H1N1 pdm vaccine. NYMC X-183 was used as the H3N2 component for the Southern Hemisphere flu vaccine formulation, 2010. NYMC X-181 and NYMC X-187 (H3N2) are being used in the Northern Hemisphere flu vaccine formulation, 2010-2011. Approximately 400-500 million doses are prepared for the Northern Hemisphere (FIGS. 7C, 7D, and 8).

Time Requirement (for Developing Reassortant Seed Virus):

Total time in developing reassortant seed virus using standard RT-PCR requires approximately 23-28 days. For example, the time for production of NYMC X-179A [used as seed strain for A(2009 H1N1)pdm vaccine] was 23 days. When the results obtained using the present Short-run RT-PCR assay (presented in FIGS. 7C, 7D, and 8) are practiced in combination with reduced incubation times of about 16-18 hrs, it is expected that the time (to development of reassortant seed virus) can be reduced to approximately 10 days. Additionally, with Short-run RT-PCR it is possible to monitor different steps more frequently and also eliminate some steps, speeding up the time to development of the reassortant(s) and thus decreasing the time to preparation of the vaccine.

What is claimed is:

1. An isolated oligonucleotide selected from the group consisting of: SEQ ID NO:1, SEQ ID NO:2, SEQ ID NO:3, SEQ ID NO: 4, SEQ ID NO: 5, SEQ ID NO:6, SEQ ID NO:7, SEQ ID NO:8, SEQ ID NO:9, SEQ ID NO:10, SEQ ID NO:11, and SEQ ID NO:12.

2. A composition comprising at least one of the isolated oligonucleotides of claim 1.

3. An isolated oligonucleotide having a maximum length of 40 nucleotides comprising an oligonucleotide selected from the group consisting of: SEQ ID NO:1, SEQ ID NO: 2, SEQ ID NO:3, SEQ ID NO: 4, SEQ ID NO: 5, SEQ ID NO:6, SEQ ID NO:7, SEQ ID NO:8, SEQ ID NO:9, SEQ ID NO:10, SEQ ID NO:11, and SEQ ID NO:12.

4. A method of detecting an Influenza A virus subtype in a biological sample, the method comprising the steps of:
(i) performing reverse transcription (RT) of a viral RNA template;
(ii) performing polymerase chain reaction (PCR) comprising (a) at least one isolated oligonucleotide selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, and (b) a counterpart oligonucleotide thereof.
(iii) analyzing the product obtained in step (ii);
wherein, the Influenza A virus is selected from the group consisting of subtypes H1N1, H3N2 and A(2009 H1N1)pdm.

5. The method of claim 4 further comprising the steps of:
(i) obtaining a biological sample; and
(ii) extracting/isolating viral RNA from the sample.

6. The method of claim 4 wherein the Influenza A virus is subtype H1N1.

7. The method of claim 4 wherein the Influenza A virus is subtype H3N2.

8. The method of claim 4 wherein the Influenza A virus subtype is A(2009 H1N1)pdm.

9. The method of claim 4 wherein steps (i) and (ii) are performed in a single reaction mixture.

10. The method of claim 4, wherein step (i) is completed in about 15 minutes to about 30 minutes.

11. The method of claim 4, wherein step (i) is completed in about 15 minutes.

12. The method of claim 4, wherein steps (i)-(iii) are completed in about 75 minutes to about 90 minutes.

13. The method of claim 4, wherein steps (i)-(iii) are completed in about 75 minutes.

14. The method of claim 4 wherein steps (i)-(iii) are completed in about 90 minutes.

15. An article of manufacture comprising
(i) a container;
(ii) a composition within the container, wherein the composition comprises (a) at least one oligonucleotide selected from the group consisting of: SEQ ID NO:1, SEQ ID NO:2, SEQ ID NO:3, SEQ ID NO:4, SEQ ID N5, SEQ ID NO:6, SEQ ID NO:7, SEQ ID NO:8, SEQ ID NO:9, SEQ ID NO:10, SEQ ID NO:11, and SEQ ID NO:12, (b) a counterpart oligonucleotide thereof.

16. The article of manufacture of claim 15 further comprising a label and/or instructions directing use of the composition for detecting an Influenza A virus subtype selected from the group consisting of subtypes H1N1, H3N2 and A(2009 H1N1)pdm.
